SEQUENCE LISTING (11 sequences)

SEQ ID NO: 1 - DNA, 23 bp, Artificial Sequence
  N-Myc primer
tgatgaagaggaagatgaacagg

SEQ ID NO: 2 - DNA, 20 bp, Artificial Sequence
  N-Myc primer
tcttgggacgcacagtgatg

SEQ ID NO: 3 - DNA, 25 bp, Artificial Sequence
  N-Myc probe
actgtggagaagcggcgttcctcct

SEQ ID NO: 4 - DNA, 23 bp, Artificial Sequence
  Twist primer
ggacaagctgagcaagattcaga

SEQ ID NO: 5 - DNA, 22 bp, Artificial Sequence
  Twist primer
tctggaggacctggtagaggaa

SEQ ID NO: 6 - DNA, 22 bp, Artificial Sequence
  Twist probe
agctggcggccaggtacatcga

SEQ ID NO: 7 - DNA, 23 bp, Artificial Sequence
  ODC primer
ctgtcgtctcagtgtgaaattcg

SEQ ID NO: 8 - DNA, 17 bp, Artificial Sequence
  ODC primer
cgcccgttccaaaagga

SEQ ID NO: 9 - DNA, 21 bp, Artificial Sequence
  5′ Target Region of the Twist Gene mRNA
aacagcgaggaagagccagac

SEQ ID NO: 10 - DNA, 25 bp, Artificial Sequence
  3′ Target Region of the Twist Gene mRNA
aagatggcaagctgcagcgctatgt

SEQ ID NO: 11 - DNA, 21 bp, Artificial Sequence
  Scramble or Random iRNA
aagcgcgctttgtaggattcg

(19) United States
(12) Patent Application Publication
(10) Pub. No.: US 2007/0134674 A1
(43) Pub. Date: Jun. 14, 2007

(54) Method for the prognosis and/or diagnosis of a cancer

(76) Inventors: Bruno Mougin, Lyon (FR); Alexander Krause, Lyon (FR); Alain Puisieux, Ruy Montceau (FR); Sandrine Valsesia-Wittmann, Chassieu (FR)

Correspondence Address: OLIFF & BERRIDGE, PLC, P.O. BOX 19928, ALEXANDRIA, VA, 22320, US

(21) Appl. No.: 10/594,584

(22) PCT Filed: Apr. 6, 2005
(86) PCT No.: PCT/FR05/50210

§ 371 Date: Sep. 27, 2006

(30) Foreign Application Priority Data
Apr. 6, 2004 (FR) 0450689

(51) Int. Cl. C12Q 1/68; G01N 33/574; A61K 38/55

(52) U.S. Cl. 435/6; 435/7.23; 514/2

(57) ABSTRACT

The present invention relates to a method for the prognosis and/or diagnosis of a cancer using a biological sample taken from a patient, according to which the expression of the twist gene is determined. The invention also relates to the use of at least one inhibitor of twist gene expression for the manufacture of a medicament for combating cancer.

The present invention relates to the twist gene, and to the use thereof in the field of the diagnosis and/or treatment of a cancer.

Cancer is one of the greatest causes of mortality in developed countries. The early detection of a cancer is essential for preventing as successfully as possible the development thereof, the patient thus has a better chance of survival and the cost of his or her treatment is potentially decreased as a result. Among the diagnostic tools which make it possible to give a prognosis for the disease and to monitor the latter, mention may be made of the analysis of the expression of certain target genes which play an essential role in the evolution of certain cancers. Thus, in the case of breast cancer, mention may be made of the target gene encoding the HER2 receptor, which is overexpressed in approximately ¼ of invasive breast cancers (Slamon et al, Science, 1987, 235: 177-182). In the case of another cancer, neuroblastoma, an emerging tumour of cells of the neural crest responsible for the formation of the sympathic nervous tissue, generally observed in children under five years of age, the prognosis can be established by studying the N-myc oncogene (Seeger et al, N Engl J Med. 1985; 313(18):1111-6; Rubie et al J Clin Oncol. 1997 March; 15(3):1171-82).

These target genes are a considerable asset in combating cancer, because they can be used as a diagnostic and/or prognostic marker for the cancer, but can also be the target of novel anticancer medicaments.

Surprisingly, the inventors have demonstrated that the twist gene, which encodes a bHLH (basic Helix Loop Helix) leucine zipper transcription factor, is very relevant in combating cancer, such as breast cancer, melanoma or neuroblastoma. Specifically, the inventors have shown that the inhibition of the expression of the twist gene makes it possible to restore the apoptotic properties of the tumour cells and the sensitivity to treatment by chemotherapy.

In this respect, the invention relates to the use of at least one inhibitor of the twist gene expression, for the manufacture of a medicament for combating cancer, preferably breast cancer, neuroblastoma or melanoma. If the activity of the twist gene is directly or indirectly blocked, the apoptotic capacities of cancer cells are then restored: the cell death thus induced makes it possible to reduce the tumour.

The invention also relates to a pharmaceutical composition comprising at least one twist inhibitor and at least one pharmaceutically acceptable excipient.

For the purpose of the present invention, the term cancer is intended to mean all diseases due to an abnormal cell multiplication; in particular, in a nonlimiting manner, myelomas, lymphomas, leukaemias, carcinomas of the kidney, brain, colon, prostate, rectum, pancreas, ovaries, lung, liver or breast, and skin cancers chosen from carcinomas, melanomas and neuroblastomas. According to a preferred embodiment of the invention, the cancer is a neuroblastoma, a melanoma or a breast cancer.

The term inhibitor of twist gene expression is intended to mean a molecule (or a set of molecules) which directly or indirectly blocks the expression of the twist gene directly or indirectly, i.e., for example, by blocking the expression of the gene or by blocking the activity of the protein encoded by this gene. By way of example of methods for blocking the expression of the gene directly, mention may be made of: interfering RNAs, antisense oligonucleotides, morpholinos, etc. By way of example of methods for blocking the expression of the gene indirectly, i.e. by blocking the activity of the protein, mention may be made of: interfering chemical molecules, aptamers, dominant negative mutants, etc.

The medicament according to the invention can be in the form of a pharmaceutical composition in combination with at least one pharmaceutically acceptable excipient well known to those skilled in the art. In the pharmaceutical compositions according to the invention, for oral, sublingual, subcutaneous, intramuscular, intravenous, topical, intratracheal, rectal or transdermal administration, the inhibitor of twist gene expression can be administered in a unit administration form or as a mixture with pharmaceutical supports which are conventional, and intended for oral administration, for example in the form of a tablet, a gelatine capsule, an oral solution, etc., or rectal administration, in the form of a suppository, parenteral administration, in particular in the form of an injectable solution, in particular intravenous, intradermal, subcutaneous, etc. administration, according to conventional protocols well known to those skilled in the art.

When a solid composition in the form of tablets is prepared, the inhibitor of twist gene expression is mixed with a pharmaceutically acceptable excipient, also called pharmaceutical carrier, such as gelatine, starch, lactose, magnesium stearate, talc, gum Arabic, or the like. The tablets can be coated with sucrose, with a cellulose derivative, or with other appropriate substances. They can also be treated such that they have a sustained or delayed activity and such that they continuously release a predetermined amount of the inhibitor of twist gene expression. It is also possible to obtain a preparation of gelatine capsules by mixing the inhibitor of twist expression with a diluent and pouring the mixture into soft or hard gelatine capsules. It is also possible to obtain a preparation in the form of a syrup or for administration in the form of drops, in which the inhibitor of twist gene expression is present together with a sweetener, an antiseptic, such as in particular methylparaben or propylparaben, and also an appropriate flavour enhancer or dye. Water-dispersible powders or granules can contain the inhibitor of twist gene expression as a mixture with dispersing agents or wetting agents, or suspending agents, well known to those skilled in the art. For parenteral administration, use is made of aqueous suspensions, isotonic saline solutions or sterile and injectable solutions which contain dispersing agents, wetting agents which are pharmacologically compatible, such as, in particular, propylene glycol or butylene glycol.

The inventors have also shown that the twist gene can be used for the prognosis and/or diagnosis of a cancer, such as, in particular, breast cancer, neuroblastoma or melanoma.

To this effect, the invention relates to a method for the prognosis and/or diagnosis, in vitro, of a cancer using a biological sample taken from a patient, according to which the expression of the twist gene is determined.

According to a preferred embodiment of the invention, this method comprises the following steps:
a. a biological sample from the patient is provided and biological material is extracted from the biological sample,
b. at least one reagent specific for the twist gene is provided,
c. the expression of the twist gene is determined.

The term biological sample is intended to mean any material derived from the patient, liable to contain a biological material which makes it possible to detect the expression of a gene, and can in particular be a blood sample, a serum sample, a saliva sample or a tissue sample derived from the taking of a tumour sample or of a sample of circulating cells from the patient. This biological sample is provided by any means of taking a sample known to those skilled in the art, such as, in particular, the taking of a blood sample or of a tissue biopsy.

According to a preferred embodiment of the invention, the biological sample taken from the patient is a tissue or blood sample.

As defined above, the term cancer is intended to mean all diseases due to an abnormal cell multiplication, in particular, in a nonlimiting manner, myelomas, lymphomas, leukaemias, carcinomas of the kidney, brain, colon, prostate, rectum, pancreas, ovaries, lung, liver or breast, and skin cancers chosen from carcinomas, melanomas and neuroblastomas. According to a preferred embodiment of the invention, the cancer is a neuroblastoma or a breast cancer.

The term biological material is intended to mean any material which makes it possible to detect the expression of a gene, such as, in particular, a nucleic acid or a protein. The nucleic acid can in particular be an RNA (ribonucleic acid) such as an mRNA (messenger RNA).

The extraction of the biological material from the biological sample in step a) can be carried out by any of the protocols for extracting nucleic acids or proteins well known to those skilled in the art.

By way of indication, the nucleic acid extraction can in particular be carried out by means of a step consisting of lysis of the cells present in the biological sample, in order to release the nucleic acids contained in the protein and/or lipid envelopes of the microorganisms (such as cell debris which disturbs the subsequent reactions). By way of example, use may be made of the lysis methods as described in patent applications WO-A-00/05338, regarding mixed magnetic and mechanical lysis, WO-A-99/53304 regarding electrical lysis, and WO-A-99/15321 regarding mechanical lysis. Those skilled in the art may use other well known methods of lysis, such as thermal or osmotic shocks or chemical lysis with chaotropic agents such as guanidium salts (U.S. Pat. No. 5,234,809).

This lysis step is then generally followed by a purification step, which makes it possible to separate the nucleic acids from the other cell constituents released in the lysis step. This step generally makes it possible to concentrate the nucleic acids. By way of example, it is possible to use magnetic particles optionally coated with oligonucleotides, by adsorption or covalence (see, in this respect, patents U.S. Pat. No. 4,672,040 and U.S. Pat. No. 5,750,338), and thus to purify the nucleic acids which are bound to these magnetic particles, by means of a washing step. This nucleic acid purification step is particularly advantageous if it is desired to subsequently amplify said nucleic acids. A particularly advantageous embodiment of these magnetic particles is described in patent applications WO-A-97/45202 and WO-A-99/35500. Another advantageous example of a method for purifying nucleic acids is the use of silica, either in the form of a column, or in the form of inert particles (Boom R. et al., J. Clin. Microbiol., 1990, no. 28(3), p. 495-503) or magnetic particles (Merck: MagPrepâ Silica, Promega: MagneSil™ Paramagnetic particles). Other very widely used methods are based on ion exchange resins in a column or a paramagnetic particulate format (Whatman: DEAE-Magarose) (Levison P R et al., J. Chromatography, 1998, p. 337-344). Another very relevant but nonexclusive method for the invention is that of adsorption onto a metal oxide support (company Xtrana: Xtra-Bind™ matrix).

The term specific reagent is intended to mean a reagent which reacts with the biological material in order to demonstrate, directly or indirectly, the expression of the twist gene, which can be determined by analysis of the mRNAs derived from this gene, or by analysis of the protein encoded by this gene.

By way of indication, when it is desired to determine the expression of the twist gene by analysis of the protein encoded by this gene, this specific reagent comprises at least one antibody specific for the protein encoded by the twist gene.

By way of indication, when it is desired to determine the expression of the twist gene by analysis of the mRNAs transcribed from this gene, this specific reagent comprises at least one amplification primer specific for the complementary DNA of this mRNA (this is then referred to as an amplification primer specific for the twist gene). The complementary DNA of an mRNA can be obtained according to a protocol well known to those skilled in the art.

By way of indication, in step a) of the method according to the invention, the total RNAs (comprising the ribosomal RNAs, the transfer RNAs and the mRNAs) are extracted from the biological sample. A reverse transcription reaction is then carried out using a reverse transcriptase enzyme which makes it possible to obtain, from an RNA fragment, a complementary DNA fragment. The realisation of such a step is well known to those skilled in the art. When it is desired more particularly to obtain only the complementary DNAs of the messenger RNAs, this enzymatic step is carried out in the presence of nucleotide fragments comprising only thymine bases (polyT), which hybridize by complementarity to the polyA sequence of the various mRNAs, so as to form a polyT-polyA complex which then serves as a starting point for the reverse transcription reaction carried out by the reverse transcriptase enzyme.

Various complementary DNAs of the various messenger RNAs initially present in the biological sample are then obtained. In the subsequent disclosure, the term “cDNA” is intended to mean a complementary DNA of a messenger RNA.

The term “amplification primer” is intended to mean a nucleotide fragment comprising from 5 to 100 nucleotide units, preferably from 15 to 25 nucleotide units, having a hybridization specificity under given conditions for the initiation of an enzymatic polymerization, for example in an enzymatic amplification reaction.

The term “enzymatic amplification reaction” is intended to mean a process generating multiple copies of a target nucleotide fragment by means of specific amplification primers through the action of at least one enzyme. Such amplification reactions are well known to those skilled in the art and mention may in particular be made of the following techniques:
PCR (Polymerase Chain Reaction), as described in patents U.S. Pat. No. 4,683,195, U.S. Pat. No. 4,683,202 and U.S. Pat. No. 4,800,159,
LCR (Ligase Chain Reaction), disclosed, for example, in patent application EP-A-0 201 184,
RCR (Repair Chain Reaction), described in patent application WO-A-90/01069,
3SR (Self Sustained Sequence Replication) with patent application WO-A-90/06995,
NASBA (Nucleic Acid Sequence-Based Amplification) with patent application WO-A-91/02818, and
TMA (Transcription Mediated Amplification) with patent U.S. Pat. No. 5,399,491.

More specifically, NASBA is a technology consisting of isothermal amplification of the nucleic acid, based on the joint action of three enzymes (AMV reverse transcriptase, Rnase-H and T7 RNA polymerase). Combined with amplification primers specific for a target sequence, it amplifies the RNA targets more than a billion times in 90 minutes. The amplification reaction is carried out at 41° C. and gives single-stranded RNA molecules as the final product. NASBA requires a pair of primers, at least one of which comprises a promoter for the initiation of transcription by a T7 bacteriophage polymerase.

The term “amplicons” is used to denote the polynucleotides generated by an enzymatic amplification technique.

Preferably, when the enzymatic amplification is a PCR, the specific reagent comprises at least 2 specific amplification primers in order to amplify a specific region of the complementary DNA of the mRNA derived from the twist gene. When the enzymatic amplification is a PCR carried out after a reverse transcription reaction, this is referred to as an RT-PCR.

The term “hybridization probe” is intended to mean a nucleotide fragment which has a hybridization specificity under given conditions so as to form a hybridization complex with a target nucleotide fragment.

The term “hybridization” is intended to mean the process during which, under appropriate conditions, two nucleotide fragments, such as, for example, a hybridization probe and a target nucleotide fragment, having sufficiently complementary sequences are capable of forming a double strand with stable and specific hydrogen bonds. A nucleotide fragment “capable of hybridizing” with a polynucleotide is a fragment which can hybridize with said polynucleotide under hybridization conditions, which can be determined in each case in a known manner. The hybridization conditions are determined by virtue of the stringency, i.e. the strictness of the operating conditions. The higher the stringency at which the hybridization is carried out, the more specific it is. The stringency is defined in particular as a function of the base composition of a probe/target duplex, and also by virtue of the degree of mismatch between two nucleic acids. The stringency can also depend on the reaction parameters, such as the concentration and the type of ion species present in the hybridization solution, the nature and the concentration of denaturing agents and/or the hybridization temperature. The stringency of the conditions under which a hybridization reaction must be carried out will depend mainly on the hybridization probes used. All these data are well known and the appropriate conditions can be determined by those skilled in the art. In general, depending on the length of the hybridization probes used, the temperature for the hybridization reaction is between approximately 20 and 70° C., in particular between 35 and 65° C. in a saline solution at a concentration of approximately 0.5 to 1 M. A step consisting of detection of the hybridization reaction is then carried out.

The term “detection” is intended to mean either direct detection by means of a physical method, or a method of detection using a label. Numerous detection methods exist for the detection of nucleic acids [see, for example, Kricka et al., Clinical Chemistry, 1999, no. 45(4), p. 453-458 or Keller G. H. et al., DNA Probes, 2nd Ed., Stockton Press, 1993, sections 5 and 6, p. 173-249]. The term “label” is intended to mean a tracer capable of engendering a signal. A nonlimiting list of these tracers comprises enzymes which produce a signal detectable, for example, by colorimetry, fluorescence or luminescence, such as horseradish peroxidase, alkaline phosphatase, beta-galactosidase or glucose-6-phosphate dehydrogenase; chromophores such as fluorescent, luminescent or dye compounds; electron dense groups which can be detected by electron microscopy or by virtue of their electrical properties such as conductivity, by amperometry or voltametry methods, or by impedance measurements; groups which can be detected by optical methods such as diffraction, surface plasmon resonance or contact angle variation, or by physical methods such as atomic force spectroscopy, tunnel effect, etc.; radioactive molecules such as 32P, 35S or 125I. Thus, the polynucleotide can be labelled during the enzymatic amplification step, for example using a labelled triphosphate nucleotide for the amplification reaction. The labelled nucleotide will be a deoxyribonucleotide in amplification systems generating a DNA, such as PCR, or a ribonucleotide in amplification techniques generating an RNA, such as the TMA or NASBA techniques. The polynucleotide can also be labelled after the amplification step, for example by hybridizing a labelled probe according to the sandwich hybridization technique described in document WO-A-91/19812.

Another preferential specific method of labelling nucleic acids is described in application FR-A-2 780 059. Another preferential method of detection uses the 5′-3′ exonuclease activity of a polymerase, as described by Holland P. M., PNAS (1991) p. 7276-7280. Signal amplification systems can be used as described in document WO-A-95/08000 and, in this case, the preliminary enzymatic amplification reaction may not be necessary.

For the purpose of the present invention, the hybridization probe may be a probe referred to as a capture probe. In this case, the target nucleotide fragment can be labelled beforehand by means of a label. The “capture” probe is immobilized or immobilizable on a solid support by any appropriate means, i.e. directly or indirectly, for example by covalence or adsorption. A hybridization reaction is then carried out between said detection probe and the labelled target nucleotide fragment.

The hybridization probe can also be a probe referred to as a detection probe. In this case, the hybridization probe can be labelled by means of a label. A hybridization reaction between said capture probe and the target nucleotide fragment is then carried out. The detection probe can also be a “molecular beacon” detection probe as described by Tyagi & Kramer (Nature biotech, 1996, 14: 303-308).

The determination of the expression of the twist gene (step c) can be carried out by any of the protocols known to those skilled in the art. This expression can be analysed through the expression of the mRNAs which are transcribed at a given moment. In this case, the biological material is a nucleic acid and the specific reagent can be, without implied preference, an amplification primer or a hybridization probe as defined above.

The expression of the twist gene can also be analysed through the expression of the proteins encoded by this gene. In this case, the biological material is a protein and the specific reagent can be an antibody specific for the protein encoded by the twist gene.

By way of indication, the expression of the twist gene can be determined in the following way:
1) after having extracted the total RNAs from a biological sample as defined in step a) of the method according to the invention as presented above, a reverse transcription step is carried out, as described above, in order to determine the various complementary DNAs of the various messenger RNAs initially present in the biological sample (or cDNA);
2) the cDNAs are specifically amplified. In this case, the specific reagent used comprises at least one amplification primer specific for the twist gene. This step can be carried out in particular by means of a PCR-type amplification reaction or by means of any other amplification technique as defined above;
3) the expression of the twist gene is determined by quantifying the cDNAs. The cDNAs can be quantified in particular using a quantification range obtained by means of an amplification reaction carried out to saturation. In order to take into account the variability in enzymatic efficiency that can be observed in the various steps (reverse transcription, PCR, etc.), the expression of the twist gene of the various groups of patients can be standardized by simultaneously determining the expression of a “housekeeping” gene, the expression of which is similar in the various groups of patients. By relating the expression of the target gene to the expression of the housekeeping gene, any variability between the various experiments is thus corrected. Those skilled in the art may refer in particular to the following publications: Bustin S A Journal of molecular endocrinology, 2002, 29: 23-39; Giulietti A Methods, 2001, 25: 386-401.

The expression of the twist gene can also be determined in the following way:
1) after having extracted the total RNAs from a biological sample as defined in step a) of the method according to the invention as presented above, a reverse transcription step is carried out, as described above, in order to obtain the various complementary DNAs of the various messenger RNAs initially present in the biological sample (or cDNA);
2) the cDNAs are immobilized on a membrane;
3) the expression of the twist gene is determined by hybridizing the cDNAs to pre-labelled hybridization probes specific for the twist gene. Such hybridization techniques are well known to those skilled in the art and mention may in particular be made of the Northern blotting technique. This hybridization reaction can be carried out after a step consisting of specific amplification of the complementary DNAs of the messenger RNAs of the twist gene.

Analysis of the expression of the twist gene then makes it possible to provide a tool for the diagnosis and/or prognosis of a cancer. It is possible, for example, to analyse the expression of the twist gene in a patient who may develop a cancer, and, by comparing this expression with known average expression values for the twist gene of normal patients and known average expression values for the twist gene of patients suffering from a cancer, to determine whether the patient is developing a cancer, in order to propose to the patient a suitable treatment.

Finally, the invention relates to a kit for the diagnosis and/or prognosis of a cancer, comprising at least one reagent specific for the twist gene, as defined above.

The following examples are given by way of illustration and are in no way limiting in nature. They will make it possible to understand the invention more clearly.

### EXAMPLE 1—EXPRESSION OF THE TWIST GENE IN NEUROBLASTOMA

1) Characteristics of the biological samples: 23 neuroblastoma samples obtained from the Centre Léon Bérard (CLB) in Lyons, France, were used in this study. These neuroblastoma samples were taken prior to any therapeutic treatment. Each tumour was classified according to the INSS classification (International Neuroblastoma Staging System; Brodeur et al; (1993) J. Clin. Oncol. 11, 1466-77). 12 stage ½ tumours, 4 stage 4s tumours and 7 stage 4 samples (2 tumour punctures, 1 biopsy, 4 massively invaded bone marrow punctures) were then distinguished. The histochemical analysis showed the presence of approximately 80% of tumour cells in the localised tumours. The immunocytochemical analysis also showed, in the bone marrow punctures, the presence of approximately 80% of tumour cells. The median age of the patients at the time of neuroblastoma diagnosis was 10 and a half months, and 5 patients died during the median follow-up period of 75 months. The patients who had died during the study and the patients presenting a stage 4 neuroblastoma were described as patients with poor prognosis (PP), while the patients who were alive, having developed a stage 1, 2 and 4s neuroblastoma, were described as patients with good prognosis (GP) (description according to Brodeur, 2003, Nat Rev Cancer, 203-216). This analysis was thus carried out on 8 PP patients and 15 GP patients.
2) Extraction of the biological material (total RNAs) from the biological sample: the total RNAs were extracted using a Trizol® buffer. After the step consisting of homogenization in 1 to 1.5 ml of Trizol buffer (Invitrogen, Cergy Pointoise, France), the homogenates were treated with 300 μl of chloroform in order to remove the protein and lipid contaminants. The RNAs were then precipitated with 750 μl of isopropanol, and washed twice in 80% (vol/vol) ethanol and resuspended in DEPC water. The total RNAs were then purified in Qiagen RNeasy columns (Qiagen, Hilden, Germany) according to the manufacturer's instructions, with the exception of the final elution, which was carried out in 200 μl of RNAse-free water after incubation at 65° C. for one minute. Before the reverse transcription step, an additional precipitation step was carried out with a solution of ammonium acetate (0.5 volumes, 7.5M) and ethanol (2.5 volumes) in order to increase the concentration and the purification of the total RNAs. The completeness and the quality of the total RNAs were verified using the Agilent 2100 bioanalyser (Agilent Technologies, Waldbronn, Germany).
3) DNA chip hybridization: in order to analyse the expression of the twist gene in the neuroblastomas of GP and PP patients, the complementary DNAs (cDNA) of the mRNAs contained in the total RNAs as purified above, were obtained from 10 μg of total RNAs using 400 units of SuperScriptII reverse transcription enzyme (Invitrogen) and 100 pmol of poly-T primer containing the T7 promoter (T7-oligo(dT)₂₄-primer, Proligo, Paris, France). The cDNAs thus obtained were then extracted with phenol/chloroform and precipitated as described above with ammonium acetate and ethanol, and redissolved in 24 μl of DEPC water. A volume of 20 μl of this purified solution of cDNA was then subjected to an in vitro transcription using a T7 RNA polymerase which specifically recognizes the T7 polymerase promoter as mentioned above. This transcription makes it possible to obtain the cRNA of the cDNA. This transcription was carried out using a Bioarray High Yield RNA Transcript Labeling Kit (Enzo Diagnostics, Farmingdale, N.Y.), which makes it possible not only to obtain the cRNA but also to incorporate biotinylated cytidine and uridine bases during the synthesis of the cRNA.

The purified cRNAs were then quantified by spectrophotometry, and the cRNA solution was adjusted to a concentration of 1 μg/μl of cRNA. The step consisting of cleavage of these cRNAs was then carried out at 94° C. for 35 min, using a fragmentation buffer (40 mM of Tris acetate, pH 8.1, 100 mM of potassium acetate, 30 mM of magnesium acetate) in order to bring about the hydrolysis of the cRNAs and obtain fragments of 35 to 200 bp. The success of such a fragmentation was verified by means of a 1.5% agarose gel electrophoresis.

Next, 10 μg of fragmented cRNAs derived from each sample were added to a hybridization buffer (Affymetrix) and 200 μl of this solution were brought into contact for 16 h at 45° C. with an expression chip (Human Genome U95Av2 GeneChip® (Affymetrix)), according to the Affymetrix protocol as described on the Affymetrix internet site (see, in particular, at the following address http://www.affymetrix.com/support/downloads/manuals/expression_s2_manual.pdf).

The results showed an overexpression (×10.8) of the twist gene in the neuroblastomas of patients with poor prognosis, compared with the expression of the twist gene in the neuroblastomas of patients with good prognoses, indicating that the twist gene has a role in neuroblastoma. The analysis also showed a correlation between the expression of the twist gene and the expression of the N-Myc gene.

### EXAMPLE 2—EXPRESSION OF THE TWIST GENE IN BREAST CANCER

1) Characteristics of the biological samples: The example presented hereinafter was carried out using 23 breast tumour samples obtained from the Polyclinique Urbain V, Avignon, France. All the tumours were obtained by means of a surgical sample taken prior to any therapy (hormone therapy, radiotherapy or chemotherapy). SBRI stage tumours, i.e. tumours with good prognosis, and SBRII/III stage tumours, i.e. tumours with poor prognosis, were then distinguished. All the tumours were tested by immunohistochemistry and exhibited oestrogen receptor alpha expression. Furthermore, the lymph nodes of the patients did not have tumour cells.
2) Extraction of the biological material (total RNAs) from the biological sample: The total RNAs were extracted from 23 tumour samples, using a Trizol® buffer. After the step consisting of homogenization in 1 to 1.5 ml of Trizol buffer (Invitrogen, Cergy Pontoise, France), the homogenates were treated with 300 μl of chloroform in order to remove the protein and lipid contaminants. The RNAs were then precipitated with 750 μl of isopropanol, and washed twice in 80% (vol/vol) ethanol and resuspended in DEPC water. The total RNAs were then purified in Qiagen RNeasy columns (Qiagen, Hilden, Germany) according to the manufacturer's instructions, with the exception of the final elution, which was carried out in 200 μl of RNAse-free water after incubation at 65° C. for one minute. Before the reverse transcription step, an additional precipitation step was carried out with a solution of ammonium acetate (0.5 volumes, 7.5M) and ethanol (2.5 volumes) in order to increase the concentration and the purification of the total RNAs. The completeness and the quality of the total RNAs were verified using the Agilent 2100 bioanalyser (Agilent Technologies, Waldbronn, Germany).
3) DNA chip hybridization: In order to analyse the expression of the twist gene in SBRI stage and SBRII/III stage breast tumours, the complementary DNAs (cDNAs) of the mRNAs contained in the total RNAs as purified above, were hybridized on expression chips (U95Av2, Affymetrix) as indicated in Example 1, paragraph 3.
The cells originating from SBRII/III stage breast tumours showed an average expression of the twist gene which was 2.3 times higher than the average expression in the SBRI stage breast tumours. These results demonstrate that tumour cells with poor prognosis are associated with overexpression of the twist gene, indicating that the twist gene has a role in breast cancer.

### EXAMPLE 3—CORRELATION OF THE EXPRESSION OF THE TWIST GENE AND OF THE EXPRESSION OF THE N-MYC GENE IN NEUROBLASTOMA

1) Characteristics of the biological samples (neuroblastoma cell culture): various cell lines were established by the Centre Léon Bérard from tumour biopsies and characterized with regard to their N-Myc gene amplification status, i.e. single copy (CLB-/SedP, -/Ga), or amplified N-Myc (-/Car, -/Pe, -/Esp, -/Br, -/Bab, -/Ber, -/Deb, -/Bar, -/Ghe, -/Bac, -/Trk, -/LS, -/Ma, -/Rem, -/Vol). The other N-Myc lines were provided, respectively, by Drs. Bieder, Kemshead and Schwab and the Gustave Roussy Institute-Paris. They are, respectively, the single copy N-Myc cells SK-N-SH, SK-N-AS and SHEP, and the amplified N-Myc cells WAC, Tet21N and IGR-N91.

These cell lines are maintained at 37° C. under 5% CO₂ in 12 ml of RPMI 1640 culture medium (GIBCO Invitrogen Corporation) supplemented with 10% decomplemented foetal calf serum (FCS), 2% glutamine and penicillin/streptomycin (10 000 U/ml; 10 000 μg/ml). The cells are subjected to the action of EDTA (Life Technologies) and re-seeded at the given density.

The MEF or MEF Imk4a-Arf−/− cells were kindly provided by Dr M. van Lohuizen. These cells are primary embryonic cells. The cells are cultured at 37° C. under 5% CO₂ in DMEM medium (Invitrogen Corporation) supplemented with 10% FCS, 2% glutamine and P/S (10 000 U/ml).
2) RNA extraction: The total RNA extraction is carried out from the dry pellets using the “TriReagent” reagent (Sigma) on a “Phase Lock Gel Heavy” column (Eppendorf). The RNA pellets obtained are resuspended in a volume of 20 μl of “RNAse-free” water and the concentrations thereof are estimated by reading the OD at 260 nm. The cDNA synthesis is carried out with the “first strand cDNA synthesis kit” (Amersham) using 1 μg of RNA diluted in 8 μl of RNAse-free water. The cDNAs obtained are denatured at 95° C. for 5 min. The cDNAs are stored at −30° C. until they are used.
3) Real-time PCR: These results were confirmed by real-time PCR. For this, the amplification was carried out on the “ABI-PRISM 7000 Sequence Detection System” device, in a volume of 25 μl, in the presence of the primers and probes presented below, at respective concentrations of 300 nM, using the “Taq-man Master Mix 2×” reaction mixture (Applied Biosystems) and according to the conditions [95° C. for 15 sec; 60° C. for 1 min]×50 cycles.

The primers and probes (Eurogentec) used are reported below.

| | |
|---|---|
| N-Myc primer | 5′ TGATGAAGAG GAAGATGAAC AGG |
| N-Myc primer | 3′ TCTTGGGACG CACAGTGATG |
| N-Myc probe | Fam-ACTGTGGAGA AGCGGCGTTC CTCCT-Tamra |
| Twist primer | 5′ GGACAAGCTG AGCAAGATTC AGA |
| Twist primer | 3′ TCTGGAGGAC CTGGTAGAGG AA |
| Twist probe | Fam-AGCTGGCGGC CAGGTACATC GA-Tamra |
| ODC primer | 5′ CTGTCGTCTC AGTGTGAAAT TCG |
| ODC primer | 3′ CGCCCGTTCC AAAAGGA |

The specificity of the amplicon obtained was confirmed by “SYBR-Green” incorporation (Applied Biosystems) on successive 50/50 dilutions of the cDNAs and the obtaining of a single peak on the dissociation curve. The primers and probes (VIC labelled in the 5′ position and TAMRA labelled in the 3′ position) of the calibrating gene (RNAseP) are provided in the “ABI PRISM RNAseP Predevelopped Taq-Man Assay Reagents” kit (Applied Biosystems).

The reaction is carried out on 1 μl of complementary DNA, i.e. 1/90^{th} of the reverse transcription volume, diluted in 4 μl of water, so as to standardize the study of the various genes and minimize the variability of the RNA extraction and cDNA synthesis steps. Two replicates of each sample are tested.
4) Northern blotting: These results were also confirmed by Northern blotting. For this, 15 μg of total RNAs are separated on a 1% agarose gel, in MOPS-formaldehyde buffer, overnight at a constant voltage of 40 V. The gel is blotted by capillary action onto a Hybond N+ membrane (Amersham) overnight in blotting buffer (20×SSC). The radioactive probes used are produced by random labelling using the “Readiprim II” system (Amersham) on 50 ng of DNA in the presence of 5 μCi of α32 dCTP. After labelling for 1 hour at 37° C., the probe is purified on a column (mini quick spin DNA columns, Roche) and denatured for 5 min at 95° C. before use. The hybridization is carried out in CHURCH buffer (1M Na₂HPO₄, 1M NaH₂PO₄, 7% SDS, 1% BSA) overnight and with shaking at 65° C. The membrane is washed with 2×SSC for 20 min, and then 5 min with 0.1×SSC/0.1% SDS and placed in a cassette with a radiographic film at −80° C. The results obtained confirmed those obtained on a DNA chip and with real-time PCR.
5) Western blotting: The inventors also analysed the twist-N-myc relationship at the protein level by Western blotting, using polyclonal antibodies directed against a C16K epitope corresponding to the N-terminal part of twist (Agrobio).

The dry pellets produced on the cell lines were resuspended in 100 to 200 μl of lysis buffer (20 mM Tris HCl, pH 7.5, 1% Triton, 0.05% SDS, 0.5% sodium deoxycholate, 150 mM Nacl) in the presence of protease inhibitors: Complete cocktail (Roche) and 0.5% PMSF (Roche). After incubation for 30 min on ice the samples were centrifuged at 12 000 rpm for 5 min at 4° C. and the supernatant containing the protein fraction was recovered. The amount of total proteins was estimated by colorimetric assay, relative to a bovine serum albumin standard curve (0; 2; 4; 8; 12; 16 and 20 μg/ml), and reading of the optical density at 595 nm.

For the study of the twist (27 kDa) and N-Myc (64 kDa) proteins, 100 μg of the total protein extracts were taken up in a final volume of 36 μl in the presence of 6 μl of 6× loading buffer (375 mM Tris-HCl (pH 6.8), 6% SDS, 10% glycerol, 30% β-mercaptoethanol, 0.06% BBP) and then loaded onto an SDS-PAGE 15% gel. After migration for 2 hours at a constant voltage of 100 V in 1×Tris-glycine-SDS buffer (SIGMA), the gel was blotted, under cooling conditions, for 1 h at 200 mA, onto a nitrocellulose membrane with a porosity of 0.2 μm, in a buffer of 1×Tris-glycine-20% methanol.

After saturation of the aspecific sites (3% BSA, 5% skimmed milk or 0.2% I-Block (Tropix)), the membranes were placed in the presence of the specific primary antibody overnight at 4° C.: (i) anti-N-Myc #556438 (Pharmingen) or #sc-142 (Tebu) in 0.5% skimmed milk; (ii) anti-Twist (anti-C16K rabbit or chicken polyclonal, Agrobio) in 0.2% I-Block. After rinses in TBS-0.1% Tween (Sigma), the membranes were incubated with the corresponding secondary antibody coupled to HRP peroxidase (DAKO) for 1 hour at ambient temperature. After rinsing, the visualization was carried out using the “ECL” luminescence visualizing kit (Amersham) and detection on Biomax film. The results confirmed at the protein level the results obtained at the mRNA level.
Conclusion: The inventors have shown that the overexpression of the twist gene is directly associated with the overexpression of N-Myc, regardless of whether or not the latter is linked to a gene amplification. This association indicates that twist expression is necessary for the survival and proliferation of cells exhibiting an overexpression of N-Myc.

### EXAMPLE 4—THE TWIST GENE IS AN ONCOGENE INVOLVED IN THE INHIBITION OF N-MYC DEPENDENT APOPTOSIS

In functional terms, the inventors demonstrated that the experimental overexpression of twist in neuroblastoma-derived lines led the cells to a state of premature senescence. This type of response is a cellular defence mechanism against the induction of an oncogene (Dimri et al., 1995; for review: Mathon and Lloyd, 2001). The cell senescence was observed by means of an X-Gal staining assay at pH 6, which is a reflection of mitochondrial enzyme hyperactivity. The cells are rinsed with 1×PBS, fixed for 5 min at ambient temperature with a solution of 1×PBS, 37% formaldehyde, 25% glutaraldehyde, and incubated in the staining solution (1×PBS, 5 mM K₃Fe(CN)₆, 5 mM K₄Fe(CN)₆, 2 mM MgCl₂, 150 mM NaCl, 100 mM phosphate buffer, pH 6, 40 mM C₆H₅O₇Na₃, 1 mg/ml X-Gal) at 37° C. overnight. After rinsing with demineralized water, the staining was evaluated under a microscope.

The inventors therefore showed that it is the cooperation between H-twist and N-Myc which was responsible for the cellular transformation, demonstrated by the acquisition of cells capable of growing in semi-liquid medium. For this, the inventors developed a cellular transformation assay for primary cells. Two types of cells were used: wild-type primary embryonic cells (MEF wt) but also MEFs derived from mice bearing a KO on the Arf gene (MEF-InK4a-Arf^{-/-}). In these cells, the InK4a gene remains functional but the Arf gene, which positively regulates p53, is non-functional. These cells were kindly provided by Dr M. van Lohuizen. These cells are cultured at 37° C. under 5% CO₂ in DMEM medium (Invitrogen corporation) supplemented with 10% FCS, 2% glutamine and P/S (10 000 U/ml).

For this, the soft agar transformation assays are carried out on an agar base at 0.75% (low melting point agarose) and a top agar at 0.45% (BMA). The cells are transfected by the calcium phosphate precipitate method and then cultured in triplicate for at least 4 to 6 weeks in the presence of the antibiotics ad hoc. The transformant clones are counted and expressed as % of colonies formed relative to the number of cells seeded.

In this model, the introduction of N-Myc or H-twist into MEF wt cells does not make it possible to transform the cells, but it is the cooperation between H-twist and N-Myc which confers on the wt cells the ability to grow in semi-liquid medium, which is a reflection of cellular transformation. The inventors showed that H-twist interfered with the p53-Arf response since the sole introduction of N-Myc brought about the transformation of MEF-InK4a-Arf^{-/-}, whereas that of H-Twist did not. The joint overexpression of N-Myc and of twist led to an increase in the size and in the number of the transformed colonies, indicating that H-twist also had to be interfering with ARF-independent mechanisms.

The inventors also showed that H-twist could inhibit the apoptotic response induced by N-Myc and that the inactivation of twist expression in amplified-N-myc cell lines made it possible to restore this N-Myc-induced apoptosis. This inactivation was carried out experimentally by RNA interference or RNAi, a phenomenon of sequence-specific post-transcriptional gene quenching induced by the introduction, into the cell, of small interfering RNAs (siRNA) of 21 to 23 nucleotides (Elbashir et al., 2001).

For this, two double-stranded interfering RNA oligonucleotides (iRNAs) targeting the 5′ or 3′ region of the mRNA of the twist gene were synthesized. The sequences of the target regions recognized by these iRNAs are, respectively: AACAGCGAGGAAGAGCCAGAC for the twist 5′ iRNA, i.e. bases 51 to 72 following the translation initiation codon; AAGATGGCAAGCTGCAGCGCTATGT for the twist 3′ iRNA, i.e. bases −97 to −76 preceding the translation stop codon. The “scramble”, or random, iRNA of sequence AAGCGCGCTTTGTAGGATTCG, was chosen so as not to hybridize any known sequence of the human genome.

The cells were transfected, using the Exgen 500 reagent, with the various iRNAs at a final concentration of 100 nM, and the apoptosis was then analysed 72 hours later by flow cytometry, either by TUNEL labelling of the fragmented DNA using the “In Situ Cell Death Detection Kit, Fluoresceine or TMR” kit (Roche), or by evaluating the sub-G1 subpopulation by analysis of the cell cycle after incorporation of propidium iodide. In the first case, 10⁶ cells per point are fixed with a 1×PBS solution containing 1% PAF for one hour at ambient temperature and permeabilized using a solution of 0.1% Triton in PBS for two minutes on ice. A positive control for apoptosis is effected by incubation of cells in the presence of DNAse (Boehringer) and a negative control is effected by incubation of the cells in the buffer alone. An enzymatic labelling in the buffer is carried out for one hour at 37° C. in a humid atmosphere and in the dark, in all the other wells. After two rinses with PBS, the cells are analysed on a FACSCAN.

In the case of the cell cycle analysis, the cells are rinsed with 5 ml of PBS/1% BSA under cold conditions, and then fixed in 5 ml of 70% ethanol, vortexing for 30 minutes in ice. After rinsing with PBS, the cells are permeabilized by adding 1 ml of 2N HCl-0.5% Triton for 30 minutes at ambient temperature. After rinsing with PBS, the cells are taken up in 1 ml of PBS containing 5 μg/ml of propidium iodide, before being analysed on a Facscan. The results obtained demonstrated that the inactivation of H-twist by interfering RNA made it possible to induce up to 32% cell death compared with the nontransfected controls or controls transfected with a random interfering RNA which showed only 4 to 5% of cells in subG1. By means of TUNEL labelling, the inventors showed that the cells in which H-twist was inactivated died by apoptosis.

The results showed that, in the neuroblastoma-derived cell lines, the N-Myc dependent apoptosis was functional but inhibited by the overexpression of H-twist and that it was possible to restore programmed cell death by inhibiting H-twist.

### EXAMPLE 5—THE TWIST GENE IS AN ONCOGENE INVOLVED IN THE INHIBITION OF APOPTOSIS OF LINES DERIVED FROM BREAST CARCINOMAS AND FROM MELANOMA

As observed for the neuroblastoma-derived lines, the inventors observed an overexpression of twist in lines derived from breast cancer and from melanoma and demonstrated that this overexpression interfered with cell death.

Thus, the inventors were able to show that the inactivation of twist expression in cell lines derived from breast cancer and from melanomas made it possible to restore the ability of these cells to die by apoptosis. This inactivation was carried out experimentally by means of RNA interference, or RNAi, a sequence-specific post-transcriptional gene quenching phenomenon, using the double-stranded interfering RNA oligonucleotides (iRNAs) described in Example 4 on neuroblastoma-derived cell lines: sequence AACAGCGAGGAAGAGCCAGAC for the twist 5′ iRNA; sequence AAGATGGCAAGCTGCAGCGCTATGT for the twist 3′ iRNA, and, as a control, the “scramble”, or random, iRNA sequence AAGCGCGCTTTGTAGGATTCG.

The breast cancer or melanocyte cells were transfected, using the lipofectamine 2000 reagent (Invitrogen), with the various iRNAs at a final concentration of 100 nM in OPTIMEM serum-free minimum medium (Invitrogen), and then the normal medium was reintroduced 4 hours after transfection. The apoptosis was analysed 72 hours later by flow cytometry, either by TUNEL labelling of the fragmented DNA using the “In Situ Cell Death Detection Kit, Fluoresceine or TMR” kit (Roche), or by evaluating the sub-G1 subpopulation by analysis of the cell cycle after incorporation of propidium iodide. In the first case, 10⁶ cell per point are fixed with a 1×PBS solution containing 1% PAF, for one hour at ambient temperature, and permeabilized using a solution of 0.1% Triton in PBS for two minutes on ice. A positive control for apoptosis is effected by incubation of cells in the presence of DNAse (Boehringer) and a negative control is effected by incubation of the cells in the buffer alone. An enzymatic labelling in the buffer is carried out for one hour at 37° C. in a humid atmosphere and in the dark, in all the other wells. After two rinses with PBS, the cells are analysed on a FACSCAN.

In the case of the cell cycle analysis, the cells are rinsed with 5 ml of PBS/1% BSA under cold conditions, and then fixed in 5 ml of 70% ethanol, vortexing for 30 minutes in ice. After rinsing with PBS, the cells are permeabilized by adding 1 ml of 2N HCl −0.5% Triton for 30 minutes at ambient temperature. After rinsing with PBS, the cells are taken up in 1 ml of PBS containing 5 μg/ml of propidium iodide before being analysed on a Facscan.

The results demonstrated that the inactivation of H-twist by RNA interference made it possible to induce 2 to 10 times more cell death compared with the nontransfected controls or the controls transfected with an interfering RNA, both for the breast cancer-derived cells and the melanoma-derived cells. By means of TUNEL labelling, the inventors showed that the cells in which H-twist was inactivated died by apoptosis.

The results showed that, in the cell lines derived from melanomas and breast carcinomas overexpressing H-twist and exhibiting no mutation of the gene encoding the p53 protein, apoptosis was functional but inhibited by the overexpression of H-twist, and that it was possible to restore programmed cell death by inhibiting H-twist.

Conclusion: all these results demonstrate the participation of the twist gene in the development of a cancer, such as breast cancer, neuroblastoma or melanoma. In particular, these results demonstrate the possibility of restoring the ability of cells to die by apoptosis when expression of the twist gene is blocked, which gene is, as a result, an excellent target gene in combating cancer.

1. (canceled)

2. Pharmaceutical composition comprising at least one inhibitor of twist gene expression or of the activity of the corresponding protein and at least one pharmaceutically acceptable excipient.

3. Method for the prognosis and/or diagnosis, in vitro, of a cancer using a biological sample taken from a patient, according to which the expression of the twist gene is determined.

4. Method for the prognosis and/or diagnosis of a cancer according to claim 3, comprising the following steps:
a. a biological sample from the patient is provided and biological material is extracted from the biological sample,
b. at least one reagent specific for the twist gene is provided,
c. the expression of the twist gene is determined.

5. Method according to claim 3, wherein the biological sample taken from the patient is a tissue or blood sample.

6. Method according to claim 3, wherein the cancer is chosen from neuroblastoma or breast cancer.

7. Kit for the diagnosis and/or prognosis of a cancer, comprising at least one reagent specific for the twist gene.

8. Method according to claim 4, wherein the biological sample taken from the patient is a tissue or blood sample.

9. Method according to claim 4, wherein the cancer is chosen from neuroblastoma or breast cancer.

10. Method according to claim 5, wherein the cancer is chosen from neuroblastoma or breast cancer.

11. Method according to claim 8, wherein the cancer is chosen from neuroblastoma or breast cancer.

12. Method of treating cancer, comprising administering to a patient in need of such treatment at least one inhibitor of twist gene expression or of the activity of the corresponding protein.
